(19) United States
(12) Patent Application Publication
(10) Pub. No.: US 2026/0026702 A1
(43) Pub. Date: Jan. 29, 2026

(54) METHOD FOR MEASURING CEREBRAL VASCULAR REACTIVITY USING HYPOXIA AS VASOACTIVE AGENT

(71) Applicant: THORNHILL SCIENTIFIC INC., North York (CA)

(72) Inventors: Olivia SOBCZYK, Etobicoke (CA); Joseph Arnold FISHER, Thornhill (CA); Ece Su SAYIN, Windsor (CA); David J. MIKULIS, Oakville (CA); James DUFFIN, Toronto (CA); Julien POUBLANC, North York (CA)

(21) Appl. No.: 19/276,713

(22) Filed: Jul. 22, 2025

Related U.S. Application Data
(60) Provisional application No. 63/674,732, filed on Jul. 23, 2024.

(51) Int. Cl. A61B 5/026; A61B 5/00; G06T 7/00

(52) U.S. Cl. CPC A61B 5/0263; A61B 5/0036; A61B 5/0042; G06T 7/0016; G06T 2207/10088; G06T 2207/30016; G06T 2207/30104

(57) ABSTRACT

A method for quantitative measurement of cerebral vascular reactivity (CVR) combines sequential gas delivery with ΔR₂*-based perfusion analysis. Sequential gas delivery imposes a first stepwise reoxygenation after a first hypoxic condition and a second stepwise reoxygenation after a second hypoxic condition. In one mode, the second hypoxic condition produces greater vasodilation than the first; in another mode both hypoxia levels are minimal and an independent vasoactive stimulus, such as hypercapnia or acetazolamide, is applied between reoxygenations. MRI gradient-echo imaging records the ΔR2* time course in a target voxel during each reoxygenation. Sigmoid fitting yields perfusion metrics including relative cerebral blood flow, relative cerebral blood volume and mean transit time. Comparison of the metrics derived from the two reoxygenations provides a numerical CVR value that can be reproduced across sessions and subjects.

### CROSS-REFERENCE TO RELATED APPLICATION(S)

This application claims the benefit of U.S. Provisional Patent Application No. 63/674,732 entitled “METHOD OF MEASURING CEREBRAL VASCULAR REACTIVITY USING HYPOXIA AS VASOACTIVE AGENT”, filed Jul. 23, 2024, the entire contents of which are incorporated herein by reference.

### FIELD

The present specification relates to hemodynamic assessment, and in particular to techniques for measuring cerebrovascular reactivity.

### BACKGROUND

Cerebrovascular reactivity is most often provoked with either intravenous acetazolamide, a carbonic-anhydrase inhibitor that acidifies blood and widens cerebral vessels over a ten- to twenty-minute interval, or with hypercapnia produced by inhaled or endogenously accumulated carbon dioxide, which acts rapidly but is difficult to reproduce precisely between and within subjects. The resulting blood-flow change is typically inferred by magnetic-resonance blood-oxygen-level-dependent (BOLD) imaging or arterial spin labelling (ASL); BOLD relies on stable cerebral oxygen consumption and blood volume, whereas ASL suffers from low signal-to-noise ratio, limited spatial and temporal resolution, and sensitivity to arterial transit-time variability. Consequently, the accuracy and repeatability of cerebrovascular-reactivity assessments remain constrained by the characteristics of these stimuli and measurement techniques.

One study attempted to use hypoxia as a vasoactive stimulus (Hannah R Johnson, Max C Wang, Rachael C Stickland, Yufen Chen, and Molly G Bright, “Toward Reliable quantification of Global Cerebrovascular Reactivity to Hypoxic Hypoxia” (2024) International Society of Magnetic Resonance Medicine, Abstract 2485). The authors used a computer-controlled gas blender to induce baseline, hypoxic (PO₂=60 mmHg), and hypercapnic respiratory states, and measured cerebral blood flow in large extracranial arteries using phase contrast MRI. While the authors attempted mathematical corrections, unintentional CO₂ changes during the hypoxia caused significant variability in the measured CVR. A further limitation was that phase contrast can only be performed in large vessels or heart valves, so no measurements were taken of capillary blood flow.

### SUMMARY

The specification provides an improved method for using hypoxia as the vasoactive stimulus in the measurement of CVR. According to the methods described herein, a precisely repeatable vasoactive stimulus can be delivered to a subject for the purpose of cerebrovascular-reactivity mapping by employing sequential gas delivery to impose controlled reductions in arterial oxygen saturation, followed by abrupt reoxygenation, while independently controlling arterial carbon dioxide levels. The cerebral blood flow is directly measured from multi-echo T₂* imaging during the reoxygenation events, which allows for direct measurements of capillary blood flow in the tissues.

In one aspect, the specification provides a method of measuring cerebral vascular reactivity in a subject including using sequential gas delivery to impose a first stepwise reoxygenation from a first hypoxic condition, using sequential gas delivery to impose a second stepwise reoxygenation from a second hypoxic condition selected to induce greater vasodilation than the first hypoxic condition, measuring a ΔR₂* time course in a target voxel responsive to the first and second stepwise reoxygenations, calculating first and second perfusion metrics based on the ΔR₂* time course measured during the first and second reoxygenations, and comparing the first perfusion metric to the second perfusion metric to determine a cerebral vascular reactivity.

In one example, imposing the first and second stepwise reoxygenations includes restoring normoxia in the subject.

In one example, imposing the first and second stepwise reoxygenations includes restoring the subject's partial arterial pressure of oxygen to between 90 mm Hg and 100 mm Hg.

In one example, the second hypoxic condition has a longer duration or lower partial arterial pressure of oxygen than the first hypoxic condition.

In one example, the partial arterial pressure of oxygen during the first and second hypoxic conditions is less than 60 mm Hg.

In one example, the partial arterial pressure of oxygen during the first and second hypoxic conditions is less than 40 mm Hg.

In one example, the partial arterial pressure of carbon dioxide is maintained during performance of the stepwise reoxygenations.

In one example, calculating the first and second perfusion metrics includes fitting a sigmoid function to the ΔR₂* time course and further basing the perfusion metric for the target voxel on the sigmoid function.

In one example, the first and second perfusion metrics include relative cerebral blood volume, and the first and second perfusion metrics are computed as the magnitude of the sigmoid function.

In one example, the first and second perfusion metrics include relative cerebral blood flow, and the first and second perfusion metrics are computed as the maximum rate of decrease in the sigmoid function.

In one example, the first and second perfusion metrics include mean transit time calculated as the ratio of relative cerebral blood volume to relative cerebral blood flow.

A further aspect of the specification provides a method of measuring cerebral vascular reactivity in a subject including using sequential gas delivery to impose a first stepwise reoxygenation from a first hypoxic condition selected to minimize vasodilation, administering a vasoactive stimulus to the subject, using sequential gas delivery to impose a second stepwise reoxygenation from a second hypoxic condition selected to minimize vasodilation, measuring a ΔR₂* time course in a target voxel responsive to the first and second stepwise reoxygenations, calculating first and second perfusion metrics based on the ΔR₂* time courses measured during the first and second reoxygenations, and comparing the first perfusion metric to the second perfusion metric to determine a cerebral vascular reactivity.

In one example, the vasoactive stimulus is carbon dioxide, the first stepwise reoxygenation is imposed under normocapnia, and the carbon dioxide stimulus is administered as hypercapnia.

In one example, imposing the first and second stepwise reoxygenations includes restoring normoxia in the subject.

In one example, calculating the first and second perfusion metrics includes fitting a sigmoid function to the ΔR₂* time course and further basing the perfusion metric for the target voxel on the sigmoid function.

In one example, the first and second perfusion metrics include relative cerebral blood volume, and the first and second perfusion metrics are computed as the magnitude of the sigmoid function.

In one example, the first and second perfusion metrics include relative cerebral blood flow, and the first and second perfusion metrics are computed as the maximum rate of decrease in the sigmoid function.

In one example, the first and second perfusion metrics include mean transit time calculated as the ratio of relative cerebral blood volume to relative cerebral blood flow.

A yet further aspect of the specification provides a system for measuring cerebral vascular reactivity in a subject that includes a sequential gas delivery device configured to impose a first stepwise reoxygenation from a first hypoxic condition and a second stepwise reoxygenation from a second hypoxic condition that induces greater vasodilation than the first hypoxic condition. The system further includes a magnetic-resonance imaging system that measures a ΔR₂* time course in at least one target voxel during the first and second stepwise reoxygenations, and a processor that calculates first and second perfusion metrics from the ΔR₂* time course and compares the perfusion metrics to determine a cerebral vascular reactivity value.

In one example, the sequential gas delivery device restores normoxia during each stepwise reoxygenation.

In one example, normoxia is restored by returning the subject's arterial partial pressure of oxygen to between 90 mmHg and 100 mmHg.

In one example, the second hypoxic condition provides greater vasodilation than the first hypoxic condition by using a longer exposure, a lower arterial partial pressure of oxygen, or a combination of both parameters.

In one example, the arterial partial pressure of oxygen during the hypoxic conditions is less than 60 mmHg.

In one example, the arterial partial pressure of oxygen during the hypoxic conditions is less than 40 mmHg.

In one example, the sequential gas delivery device maintains the subject's arterial partial pressure of carbon dioxide while imposing the stepwise reoxygenations.

In one example, the processor fits a sigmoid function to each ΔR₂* time course when calculating the first and second perfusion metrics.

In one example, each perfusion metric includes relative cerebral blood volume that is determined from the magnitude of the sigmoid function.

In one example, each perfusion metric includes relative cerebral blood flow that is determined from the maximum rate of decrease in the sigmoid function.

In one example, each perfusion metric includes mean transit time that is calculated as the ratio of relative cerebral blood volume to relative cerebral blood flow.

A further aspect of the specification provides a system for measuring cerebral vascular reactivity in a subject that includes a sequential gas delivery device configured to impose a first stepwise reoxygenation from a first hypoxic condition selected to minimize vasodilation, administer a vasoactive stimulus to the subject, and impose a second stepwise reoxygenation from a second hypoxic condition selected to minimize vasodilation. The system further includes a magnetic-resonance imaging system that measures a ΔR₂* time course in at least one target voxel during the first and second stepwise reoxygenations, and a processor that calculates first and second perfusion metrics from the ΔR₂* time course and compares the perfusion metrics to determine a cerebral vascular reactivity value.

In one example, the vasoactive stimulus is carbon dioxide; the sequential gas delivery device imposes hypercapnia during the stimulus and restores normocapnia during the second stepwise reoxygenation while maintaining normoxia.

In one example, the sequential gas delivery device restores normoxia during both stepwise reoxygenations.

In one example, the processor fits a sigmoid function to each ΔR₂* time course when calculating the first and second perfusion metrics.

In one example, each perfusion metric includes relative cerebral blood volume that is determined from the magnitude of the sigmoid function.

In one example, each perfusion metric includes relative cerebral blood flow that is determined from the maximum rate of decrease in the sigmoid function.

In one example, each perfusion metric includes mean transit time that is calculated as the ratio of relative cerebral blood volume to relative cerebral blood flow.

These together with other aspects and advantages which will be subsequently apparent, reside in the details of construction and operation as more fully hereinafter described and claimed, reference being had to the accompanying drawings forming a part hereof, wherein like numerals refer to like parts throughout.

### BRIEF DESCRIPTION OF THE DRAWINGS

The patent or application file contains at least one drawing executed in color. Copies of this patent or patent application publication with color drawings will be provided by the Office upon request and payment of the necessary fee.

The present disclosure will be described with respect to the following figures:

FIG. 1 is a schematic diagram of a system for measuring cerebral vascular reactivity according to one embodiment.

FIG. 2 is a schematic diagram of an exemplary method of measuring cerebral vascular reactivity using the system of FIG. 1, according to one embodiment.

FIG. 3 is a graph representing exemplary performance of the method of FIG. 2.

FIG. 4 is a graph representing exemplary performance of the method of FIG. 2.

FIG. 5 is a graph showing transcranial Doppler measurements of vascular responses to hypoxia.

FIG. 6 is a graph representing exemplary performance of the method of FIG. 2.

FIG. 7 is a graph illustrating exemplary performance of the method of FIG. 2.

FIGS. 8A to 8D are perfusion maps representing exemplary performance of the method of FIG. 2 shown in grayscale.

FIGS. 9A to 9D are perfusion maps representing exemplary performance of the method of FIG. 2 shown in color.

### DETAILED DESCRIPTION

The following abbreviations are used herein:

| | |
|---|---|
| AIF | arterial input function |
| a.u. | arbitrary units |
| ASL | arterial spinning labelling |
| BOLD | blood oxygen level dependent imaging |
| G1 | first gas |
| G2 | second gas |
| FRC | functional residual capacity |
| MCA | middle cerebral artery |
| MRI | magnetic resonance imaging |
| MTT | mean transit time |
| PₐCO₂ | arterial partial pressure of carbon dioxide |
| PₐO₂ | arterial partial pressure of oxygen |
| PCA | posterior cerebral artery |
| PCO₂ | partial pressure of carbon dioxide |
| PO₂ | partial pressure of oxygen |
| P_{ET}CO₂ | end tidal partial pressure of carbon dioxide |
| P_{ET}O₂ | end tidal partial pressure of oxygen |
| rBAT | relative blood arrival time |
| rCBF | relative cerebral blood flow |
| rCBV | relative cerebral blood volume |
| ΔR₂* | change in the effective transverse relaxation rate (inverse of the |
| | T2* signal) |
| S | ΔR₂* signal in a voxel |
| SaO₂ | arterial blood-oxygen saturation |
| TCD | transcranial doppler |
| TE | echo time |
| TR | repetition time |

The following definitions are used herein:

“About” herein refers to a range of ±20% of the numerical value that follows. In one example, the term “about” refers to a range of ±10% of the numerical value that follows. In another example, the term “about” refers to a range of ±5% of the numerical value that follows.

“Hypoxic” herein refers to blood with abnormally low oxygen levels. Generally, a hypoxic PₐO₂ is below about 80 mmHg.

“Normoxic” herein refers to blood with normal oxygen levels. Generally, a normoxic PₐO₂ is between about 70 mmHg and about 110 mmHg.

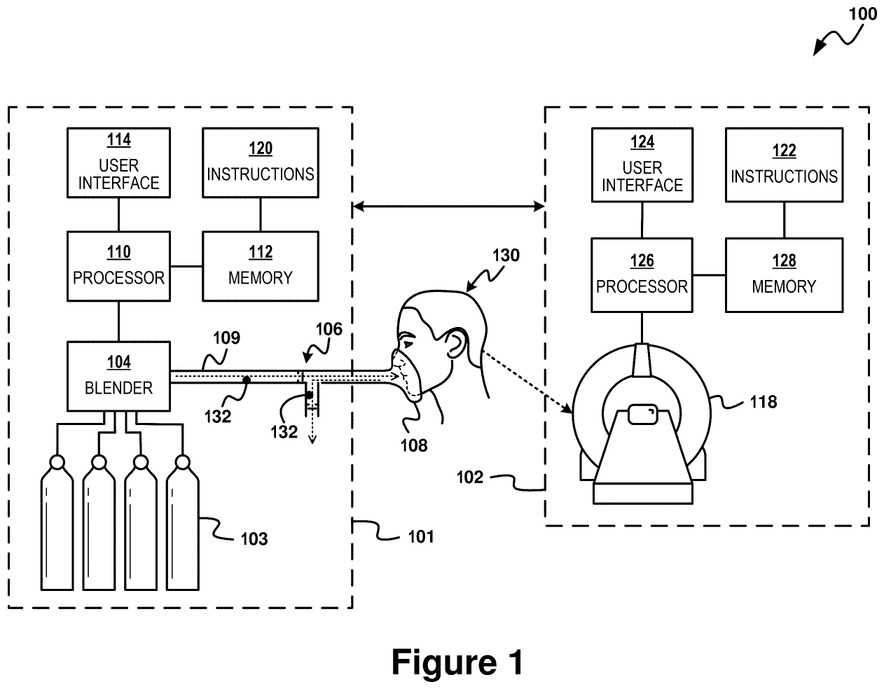

FIG. 1 shows a system 100 for measuring cerebral vascular reactivity using sequential gas delivery. The system 100 includes a respiratory device. Generally, the respiratory device comprises a means of delivering a hypoxic gas to a subject and subsequently delivering an oxygenated gas to the subject. In one example, the respiratory gas comprises an inspiratory limb with a three-way valve for delivering gas to the subject and an expiratory limb for receiving exhaled gases. The inspiratory limb is configured to provide a hypoxic gas to the subject. After inducing hypoxia in the subject, three-way valve is actuated to provide only oxygen or an oxygen-enriched gas to the subject, which generates higher hemoglobin saturation. In the examples described herein, the respiratory device is a sequential gas delivery (SGD) device 101 configured to provide gases to a subject 130 and target an arterial partial pressure of a gas such as CO₂ or O₂. Using the SGD device 101, targeted PₐO₂ values may be attained while maintaining normocapnia. The system 100 further includes a magnetic resonance imaging (MRI) system 102. The SGD device 101 includes gas supplies 103, a gas blender 104, a mask 108, a processor 110, memory 112, and a user interface 114. The SGD device 101 may be configured to control the subject's end-tidal partial pressure of CO₂ (P_{ET}CO₂) and the subject's end-tidal partial pressure of O₂ (P_{ET}O₂) by generating predictions of gas flows to actuate target end-tidal values. The SGD device 101 may be an RespirAct™ device (Thornhill Medical™: Toronto, Canada) specifically configured to implement the techniques discussed herein. For further information regarding sequential gas delivery, U.S. Pat. No. 8,844,528, US Publication No. 2018/0043117, and U.S. Pat. No. 10,850,052, which are incorporated herein by reference, may be consulted.

The gas supplies 103 may provide carbon dioxide, oxygen, nitrogen, and air, for example, at controllable rates, as defined by the processor 110. A non-limiting example of the gas mixtures provided in the gas supplies 103 is:
a. Gas A: 4% O₂, 96% N₂;
b. Gas B: 4% O₂, 96% CO₂; %
c. Gas C: 100% O₂; and
d. Calibration gas: 10% O₂, 9% CO₂, 81% N₂.

The gas blender 104 is connected to the gas supplies 103, receives gases from the gas supplies 103, and blends received gases as controlled by the processor 110 to obtain a gas mixture, such as a first gas (G1) and a second gas (G2) for sequential gas delivery.

The second gas (G2) is a neutral gas in the sense that it has about the same composition as the gas exhaled by the subject 130, which includes about 4% to 5% carbon dioxide. In some examples, the second gas (G2) may include gas actually exhaled by the subject 130. The first gas (G1) has a composition of oxygen that is equal to the target P_{ET}O₂ and preferably no significant amount of carbon dioxide. For example, the first gas (G1) may be air (which typically has about 0.04% carbon dioxide), may consist of 21% oxygen and 79% nitrogen, or may be a gas of similar composition, preferably without any appreciable CO₂.

The processor 110 may control the gas blender 104, such as by electronic valves, to deliver the gas mixture in a controlled manner. The processor 110 may be configured to compute the compositions of the first gas (G1) and the second gas (G2) required to attain the target P_{ET}O₂ and the target P_{ET}CO₂. The processor 410 may compute the compositions of the first gas (G1) and the second gas (G2) according to a prospective targeting algorithm. The processor 410 may further compute the compositions of the first gas (G1) and the second gas (G2) according to feedback received from one or more sensors 132. In particular, the sensors 132 may measure the composition of an exhaled gas.

The mask 108 is connected to the gas blender 104 and delivers gas to the subject 130. The mask 108 may be sealed to the subject's face to ensure that the subject 130 only inhales gas provided by the gas blender 104 to the mask 108. In some examples, the mask is sealed to the subject's face with skin tape such as Tegaderm™ (3M™: Saint Paul, Minnesota). A valve arrangement 106 may be provided to the SGD device 101 to limit the subject's inhalation to gas provided by the gas blender 104 and limit exhalation to the room. In the example shown, the valve arrangement 106 includes an inspiratory one-way valve from the gas blender 104 to the mask 108, a branch between the inspiratory one-way valve and the mask 108, and an expiratory one-way valve at the branch. Hence, the subject 130 inhales gas from the gas blender 104 and exhales gas to the room.

The subject 130 may breathe spontaneously or be mechanically ventilated.

The gas supplies 103, gas blender 104, and mask 108 may be physically connectable by a conduit 109, such as tubing, to convey gas. Any suitable number of sensors 132 may be positioned at the gas blender 104, mask 408, and/or conduits 409 to sense gas flow rate, pressure, temperature, and/or similar properties and provide this information to the processor 110. Gas properties may be sensed at any suitable location, so as to measure the properties of gas inhaled and/or exhaled by the subject 130.

The processor 110 may include a central processing unit (CPU), a microcontroller, a microprocessor, a processing core, a field-programmable gate array (FPGA), an application-specific integrated circuit (ASIC), or a similar device capable of executing instructions. The processor 110 may be connected to and cooperate with memory 112 that stores instructions and data.

The memory 112 includes a non-transitory machine-readable medium, such as an electronic, magnetic, optical, or other physical storage device that encodes the instructions. The medium may include, for example, random access memory (RAM), read-only memory (ROM), electrically erasable programmable read-only memory (EEPROM), flash memory, a storage drive, an optical device, or similar.

The user interface 114 may include a display device, touchscreen, keyboard, speaker, microphone, indicator, buttons, the like, or a combination thereof to allow for operator input and/or output.

Instructions 120 may be provided to carry out the functionality and methods described herein. The instructions 120 may be directly executed, such as a binary file, and/or may include interpretable code, bytecode, source code, or similar instructions that may undergo additional processing to be executed. The instructions 120 may be stored in the memory 112.

The system 100 further includes an MRI system 102 for conducting magnetic resonance imaging on the subject 130. A suitable MRI device may include a scanner 118 such as a 3-tesla (3T) MRI scanner or a 7-tesla (7T) MRI scanner. A suitable example of a 3T MRI scanner is the Signa HDxt 3.0™, provided by GE Healthcare (Milwaukee, USA). A suitable example of 7-tesla MRI scanner is the MAGNETOM™ 7T MRI, provided by Siemens (Munich, Germany). In addition to the scanner 418, the MRI system 402 may further include a processor 126, a memory 128, and a user interface 124.

Any description of the processor 126 may apply to the processor 110 and vice versa. Likewise, any description of the memory 128 may apply to the memory 112 and vice versa. Similarly, any description of the instructions 122 may apply to the instructions 120 and vice versa. Also, any description of user interface 124 may apply to user interface 114, and vice versa. In some implementations, the MRI system 102 and the SGD device 101 share one or more of a memory, processer, user interface, and instructions, however, in the present disclosure, the MRI system 102 and the SGD device 101 will be described as having respective processors, user interfaces, memories, and instructions. The processor 410 of the SGD device 101 may transmit data and instructions to the processor 126 of the MRI system 102. The processor 126 of the MRI system 102 may transmit data and instructions to the processor 110 of the SGD device 101. The system 100 may be configured to synchronize MRI imaging obtained by the MRI system 102 with measurements obtained by the SGD device 101.

The processor 126 may retrieve operating instructions 122 from the memory 128 or from the user interface 124. The operating instructions 122 may include image acquisition parameters. The parameters may include a pre-determined number of contiguous slices, a defined isotropic resolution, a diameter for the field of view, a repetition time (TR), and an echo time. Various protocols may be employed such as multi-echo T2* (ME-T₂) imaging. According to a non-limiting example of multi-echo T2* parameters, the voxel resolution is 3 mm×3 mm×3 mm, the repetition time (TR) is 1100 ms, the first echo time (TE1) is 10.7 ms, the second echo time (TE2) is 272 ms, and the third echo time (TE3) 43.6 ms.

The user interface 124 may include a display device, touchscreen, keyboard, speaker, indicator, microphone, buttons, the like, or a combination thereof to allow for operator input and/or output. Data generated and images acquired by the processor 126 may be displayed at the user interface 124.

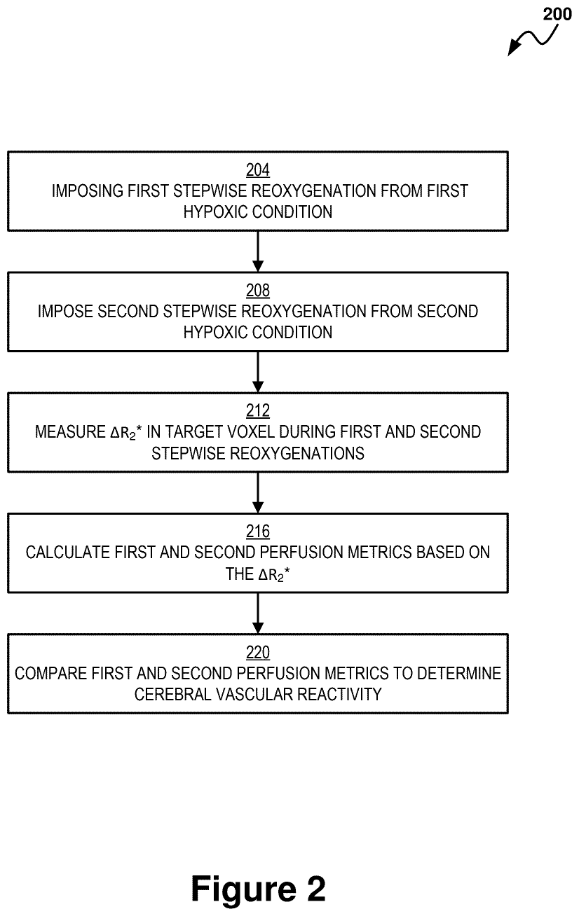

FIG. 2 shows an example method 200 of measuring cerebral vascular reactivity in a subject. The method 200 may be performed using the system 100, however the method 200 is not particularly limited.

Block 204 comprises imposing a first stepwise reoxygenation using sequential gas delivery. In system 100, block 204 is performed by the SGD device 101 which delivers gases to the subject to induce a first hypoxic condition and then reoxygenate the subject's arterial blood.

The first hypoxic condition is imposed by controlling the subject's arterial partial pressure of oxygen (PaO₂), and particularly by lowering the subject's PaO₂ below normoxia. In certain non-limiting examples, the PaO₂ of the first hypoxic condition is less than 60 mmHg, and more particularly between about 40 and about 50 mmHg. In further non-limiting examples, the PaO₂ of the first hypoxic condition is less than or about 40 mmHg. By lowering the subject's PaO₂ below 60 mmHg, block 204 can induce a measurable degree of vasodilation in the tissues. In particular, 40 mmHg is adjacent to the steepest part of the oxyhemoglobin dissociation curve and therefore, the closer the PaO₂ is to 40 mmHg, the greater the signal.

Once the first hypoxic condition is imposed for the selected duration, the first stepwise reoxygenation is imposed. The first stepwise reoxygenation comprises an increase in the subject's PaO₂ sufficient to induce a measurable magnetic signal. In some examples, the first stepwise reoxygenation restores normoxia in the subject. In further examples, the first stepwise reoxygenation restores a PaO₂ of about 80 mmHg. In further examples, the first stepwise reoxygenation restores a PaO₂ of about 85 mmHg. In further examples, the first stepwise reoxygenation restores a PaO₂ of about 90 mmHg. In further examples, the first stepwise reoxygenation restores a PaO₂ of about 95 mmHg. In further examples, the first stepwise reoxygenation restores a PaO₂ of about 100 mmHg. In further examples, the first stepwise reoxygenation restores a PaO₂ of about 105 mmHg. In yet other examples, the first stepwise reoxygenation restores a PaO₂ of between about 90 and about 100 mmHg. In some examples, the stepwise reoxygenation is abrupt, and in particular examples, the stepwise reoxygenation occurs within one inspiration. The duration of an inspiration is commonly between about 0.5 seconds and about 2.0 seconds. Generally, restoring normoxia is faster and more repeatable than targeting a hyperoxic PaO₂, and therefore this step is better tolerated by the subject, especially when blocks 204 to 212 are repeated to obtain multiple measurements.

As part of block 204, the SGD device 101 may maintain the subject's partial arterial pressure of carbon dioxide (PaCO₂) while imposing the first hypoxic condition and the first stepwise reoxygenation.

Block 208 comprises imposing a second stepwise reoxygenation using sequential gas delivery. In system 100, block 208 is performed by the SGD device 101 which delivers gases to the subject to induce a second hypoxic condition and then reoxygenate the subject's arterial blood.

Once the second hypoxic condition is imposed for the selected duration, the first stepwise reoxygenation is imposed. The second stepwise reoxygenation comprises an increase in the subject's PaO₂ sufficient to induce a measurable magnetic signal. In some examples, the second stepwise reoxygenation restores normoxia in the subject. In further examples, the second stepwise reoxygenation restores a PaO₂ of about 80 mmHg. In further examples, the second stepwise reoxygenation restores a PaO₂ of about 85 mmHg. In further examples, the second stepwise reoxygenation restores a PaO₂ of about 90 mmHg. In further examples, the second stepwise reoxygenation restores a PaO₂ of about 95 mmHg. In further examples, the second stepwise reoxygenation restores a PaO₂ of about 100 mmHg. In further examples, the second stepwise reoxygenation restores a PaO₂ of about 105 mmHg. In yet other examples, the second stepwise reoxygenation restores a PaO₂ of between about 90 and about 100 mmHg. In some examples, the stepwise reoxygenation is abrupt, and in particular examples, the stepwise reoxygenation occurs within one inspiration. The duration of an inspiration is commonly between about 0.5 seconds and about 2.0 seconds. Generally, restoring normoxia is faster and more repeatable than targeting a hyperoxic PaO₂, and therefore this step is better tolerated by the subject, especially when blocks 204 to 212 are repeated to obtain multiple measurements.

In order to measure the subject's CVR, the second hypoxic condition is selected to induce greater vasodilation than the first hypoxic condition. In particular, the duration or oxygen levels or a combination of both parameters are selected to induce more a greater vasodilatory response at block 208 than block 204. Generally, a shorter duration will minimize vasodilation and vice versa. Similarly, a higher PaO₂ will minimize vasodilation, and vice versa. The first hypoxic condition may be selected to minimize vasodilation, while the second hypoxic condition may be selected to induce vasodilation. It should be understood that blocks 204 and 208 can be performed in any order, and in some examples, the first hypoxic condition is selected to induce vasodilation, while the second hypoxic condition is selected to minimize vasodilation.

In some examples, the duration of the second hypoxic condition is the same or greater than the duration of the first hypoxic condition.

In particular non-limiting examples, the duration of the first hypoxic condition is less than six breaths. In further non-limiting examples, the duration of the first hypoxic condition is 4 to 5 breaths. In further non-limiting examples, the duration of the first hypoxic condition is less than 60 seconds. In further non-limiting examples, the duration of the first hypoxic condition is less than 30 seconds. In further non-limiting examples, the duration of the first hypoxic condition is less than 20 seconds. In further non-limiting examples, the duration of the first hypoxic condition is less than 10 seconds. In further non-limiting examples, the duration of the first hypoxic condition is between about 5 and about 30 seconds.

In particular non-limiting examples, the duration of the second hypoxic condition is more than 6 breaths. In further non-limiting examples, the duration of the second hypoxic condition is between about 12 breaths and about 20 breaths. In further non-limiting examples, the duration of the second hypoxic condition is between about 60 seconds and 120 seconds. In further non-limiting examples, the duration of the second hypoxic condition is about 60 seconds. In further non-limiting examples, the duration of the second hypoxic condition is about 90 seconds. In further non-limiting examples, the duration of the second hypoxic condition is about 120 seconds.

In some examples, the PaO₂ of the second hypoxic condition is the same or less than the PaO₂ of the first hypoxic condition.

In particular non-limiting examples, the PaO₂ of the first hypoxic condition is less than 60 mmHg. In further non-limiting examples, the PaO₂ of the first hypoxic condition is less than 50 mmHg. In further non-limiting examples, the PaO₂ of the first hypoxic condition is between about 40 mmHg and about 50 mmHg. In further non-limiting examples, the PaO₂ of the first hypoxic condition is less than or about 40 mmHg. By lowering the subject's PaO₂ below 60 mmHg, block 208 can induce a measurable degree of vasodilation in the tissues. In particular, 40 mmHg is adjacent to the steepest part of the oxyhemoglobin dissociation curve and therefore, the closer the PaO₂ is to 40 mmHg, the greater the signal.

In particular non-limiting examples, the PaO₂ of the second hypoxic condition is less than 60 mmHg. In further non-limiting examples, the PaO₂ of the second hypoxic condition is less than 50 mmHg. In further non-limiting examples, the PaO₂ of the second hypoxic condition is between about 40 mmHg and about 50 mmHg. In further non-limiting examples, the PaO₂ of the second hypoxic condition is less than or about 40 mmHg.

Generally, the parameters of the first hypoxic condition and the second hypoxic condition are not particularly limited as long as the first and second hypoxic condition induce contrasting degrees of vasodilation which can be compared to determine the subject's CVR.

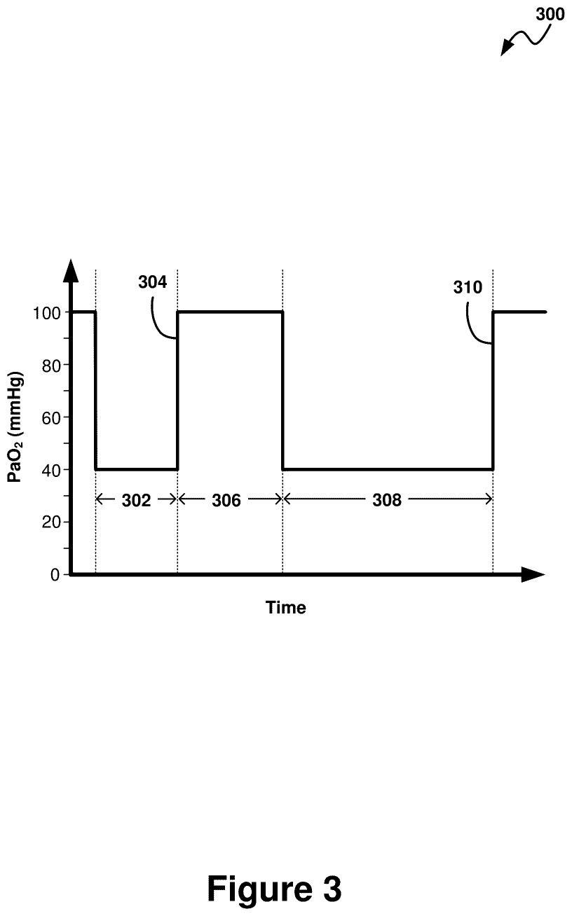

FIG. 3 is a graph illustrating exemplary performance of blocks 204 and 208. In FIG. 3, the subject's PaO₂ is plotted against time. At block 204, the SGD device 101 imposes a first hypoxic condition 302 followed by a first stepwise reoxygenation 304. In this example, the first hypoxic condition 302 is brief, however in other examples, the first hypoxic condition 302 is characterized by a higher PaO₂ selected to minimize vasodilation. In this example, the first stepwise reoxygenation 304 is selected to restore normoxia 306, and more particularly the first stepwise reoxygenation 304 restores the PaO₂ to 100 mmHg. At block 210, the SGD device 101 imposes a second hypoxic condition 308 followed by a second stepwise reoxygenation 310. In this example, the second hypoxic condition 308 is prolonged in comparison to the duration of the first hypoxic condition 302, however in other examples, the second hypoxic condition 308 is characterized by a lower PaO₂ selected to induce vasodilation.

As part of block 208, the SGD device 101 may maintain the subject's partial arterial pressure of carbon dioxide (PaCO₂) while imposing the second hypoxic condition and the second stepwise reoxygenation.

Block 212 comprises a measuring ΔR₂* time course in a target voxel responsive to the first and second stepwise reoxygenations. In system 100, block 212 is performed by the MRI system 102 which measures magnetic signals in the subject 130 while the SGD device 101 is controlling the subject's PaO₂.

As part of block 212, the MRI system 102 uses susceptibility imaging to measure T2*-weighted signals in the subject 130 during each of the stepwise reoxygenations and calculates a first and second ΔR₂* based on the respective T2*-weighted signals. For exemplary purposes, the method 200 may be explained herein with respect to a T2*-weighted signal measured in one target voxel, however it should be understood that the MRI system 102 generally measures a plurality of T2*-weighted signals in a plurality of voxels, including the target voxel. The MRI system 102 may measure the T2*-weighted signals by performing a T2*-weighted scan of the subject 130. The parameters of the T2*-weighted scan may include TR=1500 ms, TE=30 ms, flip angle=73°, 29 slices, voxel size=3 mm isotropic with 64×64 matrix, however the parameters of the T2*-weighted scan are not particularly limited and other parameters may be suitable. Because block 212 applies susceptibility imaging, measurements can be obtained from both vasculature and tissues in an area of interest.

As a further part of block 212, the processor 126 may preprocess the T2*-weighted signals. Preprocessing may include volume registering the T2*-weighted signals. Preprocessing may further include slice-time correcting the T2*-weighted signals. Preprocessing may further include co-registering the T2*-weighted signals to anatomical images. Preprocessing may further include removing noise from the T2*-weighted signals. Preprocessing may further include applying a spatial blur to the T2*-weighted signals. In particular examples, the processor 126 applies AFNI software to co-register the T2*-weighted signals to anatomical images (National Institutes of Health, Bethesda, Maryland, Version AFNI_24.0.12 ‘Caracalla’ URL https://afni.nimh.nih.gov).

As a further part of block 212, the processor 126 derives the ΔR₂* based on the T2*-weighted signal. The T2*-weighted signal may be computed into ΔR₂* using Equation 1:

$$Δ {R}_{2}^{*} ( t ) = ( \frac{- 1}{TE} ) \times ln \{ \frac{S ( t )}{S ( 0 )} \} \qquad ( 1 )$$

Since the MRI system 402 measures the magnetic signal while the respiratory device is inducing the stepwise change, block 508 produces a time course of ΔR₂* values for the selected voxel.

Block 216 comprises calculating a first and second perfusion metric based on the respective ΔR₂* values measured at block 212. In system 100, block 216 is performed by the processor 126 which retrieves a sigmoid function from memory and optimizes parameters of the sigmoid function to reduce error between the function and the ΔR₂* values.

The sigmoid function may include one or more parameters defining its amplitude, inflection point, slope, and offset. The optimization may be performed using a curve fitting algorithm, such as least squares minimization.

In some examples, the sigmoid function is symmetrical. In particular examples, the sigmoid function is a Gompertz fit function. The Gompertz fit function may be defined using Equation 2:

$${S}_{fit} ( t ) = {S}_{base} + {ae}^{- {be}^{- ct}} \qquad ( 2 )$$

$$where :$$

$$S = Δ {{R}_{2}}^{*}$$

$$t = time$$

$${S}_{fit} ( t ) = the fitted Δ {{R}_{2}}^{*} signal time course of the step response$$

$$exp = power of e$$

$${S}_{base} = the initial value of {S}_{fit} ( t )$$

$$a = the magnitude of the S decrease$$

$$b = the displacement along the time axis$$

$$c = the rate of change$$

In some examples, the sigmoid function is fitted to a portion of the ΔR₂* values derived at block 212. As part of block 216, the processor 126 may select the ΔR₂* values that coincide with the stepwise increase in PaO₂. The portion of the ΔR₂* values may be selected based on user inputs received at the user interface 124.

Block 216 further includes computing a perfusion metric based on the sigmoid function. The perfusion metric may include one or more of rCBV, rCBF, MTT, and rBAT, however the perfusion metric is not particularly limited.

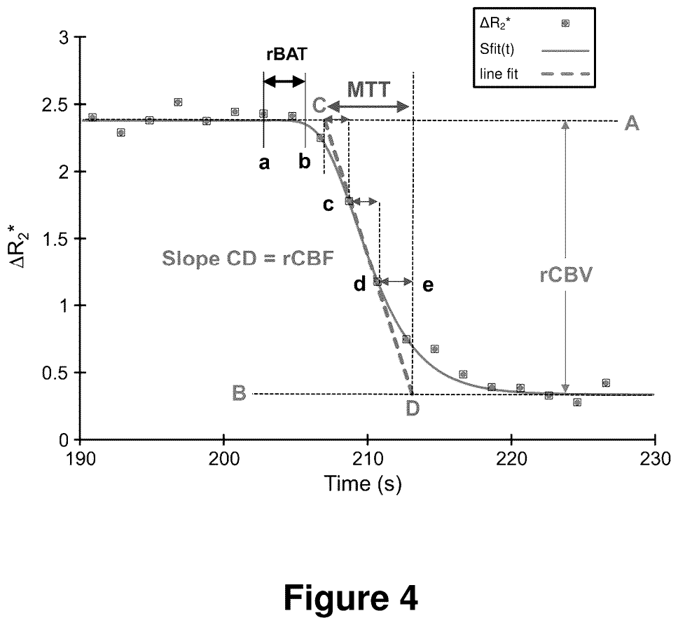

FIG. 4 is a graph illustrating exemplary performance of blocks 204 to 212. In FIG. 4, the ΔR₂* is plotted against time. The solid line shows the sigmoid function, which in this example is a Gompertz fit function fitted to the ΔR₂*. The amplitude of the Gompertz fit function is defined by line A and line B. Line CD is a tangent line at the inflection point of the sigmoid function, and the slope of line CD is the maximum rate of decrease in the sigmoid function. The mean transit time (MTT) can be calculated as the time range of the tangent line. The relative cerebral blood volume (rCBV) can be calculated as the amplitude of the sigmoid function. The relative cerebral blood flow (rCBF) can be calculated as the slope of the tangent line or the maximum rate of decrease in the sigmoid function. Reference time (a) corresponds to a time when the ΔR₂* begins to decrease in response to the stepwise increase in PaO₂. Start time (b) indicates where the ΔR₂* begins to decrease by 2% of the rCBV. The relative blood arrival time (rBAT) can be calculated as the difference between the start time (b) and the reference time (a), with negative values signifying earlier arrival.

The maximum rate of decrease of the ΔR₂* may be calculated from the Sfit (t) parameters as “a×c/e” to measure rCBF, where e is the base of natural logarithms. A tangent line with this slope is drawn through the time of maximum slope, “ln(b)/c” (FIG. 4 at CD). The tangent line defines three temporal regions, as indicated by the arrows in FIG. 4. First, the exponential increase in the rate of decline of the ΔR₂* as the step change in SaO₂ arrives at the voxel until the change has entered the voxel in all capillaries; second, a linear portion of the ΔR₂* decline as all vessels fill with the change in SaO₂ until the change begins to leave the voxel; third, an exponential decay in the rate of decline of the ΔR₂* as the SaO₂ change leaves the voxel. MTT is the sum of the time constants of the first and third temporal regions plus the time taken in the second linear ΔR₂* decrease temporal region. Consequently, MTT satisfies the central volume theorem as the ratio of CBV/CBF. Values of rCBV and rCBF were respectively multiplied by 2 and 200 to obtain easily readable values within the range of absolutes measures.

Block 220 comprises comparing the first and second perfusion metrics to determine the cerebral vascular reactivity (CVR). In system 100, block 220 is performed by the processor 126 which compares the first and second perfusion metrics.

An example of a suitable calculation is shown in Equation 3. In the example shown in Equation 3, the perfusion metric is CBF, though it should be understood that any suitable perfusion metric may be used to calculate the CVR. In Equation 3, CBF_{baseline} represents the CBF calculated at block 216 from the ΔR₂* measured during the first stepwise reoxygenation, and CBFₛₜᵢₘ represents the CBF calculated at block 216 from the ΔR₂* measured during the second stepwise reoxygenation.

$$CVR = {CBF}_{stim} - {CBF}_{baseline} \qquad ( 3 )$$

As part of block 220, the processor 126 may generate one or more perfusion maps comprising the CVR for a plurality of voxels. In particular embodiments, the processor 126 transforms the perfusion map into Montreal Neurological Institute (MNI) space and overlays the perfusion map onto their respective anatomical images.

As a further part of block 220, the processor 126 may compare the CVR of the subject to a statistical value for a reference population.

In some examples, the reference population comprises a healthy group of subjects selected exhibiting no chronic illness or disease. In further examples, the reference population comprises a group of subjects exhibiting a health condition or disease. In yet further examples, the reference population comprises a group of subjects receiving a treatment. In some examples, the comparison may be repeated by comparing the subject to two or more reference populations, for example a diseased population and a healthy population. It should be understood that the statistical value for the reference population is generated by performing blocks 204 to 216 on the group of subjects in the reference population and then combining the CVRs generated for the reference population to obtain the statistical value. In non-limiting examples, the statistical value is an average of the CVRs generated for the reference population. It should be further understood that the comparison is most effective if the same or similar parameters are employed to generate the statistical value, for instance, the CVR for the subject and the statistical value for the reference population should be obtained from measurements on corresponding voxels.

As a further part of block 220, the processor 126 may calculate a z-score representing the comparison between the perfusion metric for the subject and the statistical value for the reference population. The z-score for a plurality of voxels can be mapped to an anatomical image to obtain a z-score map.

In some examples, method 200 further includes drawing an interference based on the comparison between the subject and the reference population. The processor 126 may be configured to assess a health condition or treatment based on the comparison to the reference population.

The health condition may include a cardiovascular disease or neurological disease selected from: Parkinson's disease, stroke, hemangiomas, vascular tumor or cyst, coronary heart disease, Moyamoya disease, Cerebral Venous Thrombosis, Arteriovenous Malformation, arterio-venous fistulas, angioma formation, carotid artery disease, intracranial hypertension, steno-occlusive disease, and kidney insufficiency, however the health condition is not particularly limited. In some alternatives, the processor 126 may diagnose the health condition based on the z-score.

The treatment may include vasodilators, vasoconstrictors, anti-angiogenic agents, thrombolytics, chemotherapeutic, surgical procedures, intermittent hypoxia, exercise, diet, hydration, radiation therapy, brain stimulation, and neuromodulation, however the treatment is not particularly limited.

The diagnosis or assessment may be output at the user interface 124.

Blocks 204 to 220 may be repeated to obtain repeat measurements for a subject.

In view of the above, it will now be apparent that variants, combinations, and subsets of the foregoing embodiments are contemplated. For example, while the first hypoxic condition has been described as the baseline condition, and the second hypoxic condition has been described as the vasoactive stimulus, it should be understood that the stepwise reoxygenations may be used only for measuring the perfusion metrics, and other vasoactive stimuli may be administered to the subject. In one example, the baseline is normocapnia and the stimulus is hypercapnia. In another example, the vasoactive stimulus is an injection of acetazolamide (ACZ).

In a further variation, the ΔR₂* measured during the first reoxygenation and the ΔR₂* measured during the second reoxygenation are used as arterial input functions (AIF) which are deconvolved and used to calculate CBF (cerebral blood flow), CBV (cerebral blood volume), and MTT (mean transit time).

It will now be apparent to a person of skill in the art that the present specification affords many advantages over the prior art, and in particular, provides distinct improvements over Johnson et al. (2024).

First, Johnson reported not being able to control PCO₂ independently of PO₂ during hypoxia and having to make mathematical corrections for the lack of CO₂ control. As such, the authors mixed hypoxia and hypercapnia as opposed to hypoxia alone. In contrast, the present specification provides a method of controlling PCO₂ independently from PO₂.

Second, their target hypoxia was 60 mmHg which corresponds to SaO₂ of between 85-90%. This is very little desaturation and little effect on vasodilation. The inexact control of arterial PCO₂ confounds data related to changes in cerebral blood flow resulting from hypoxia. In contrast, the present our method reduces the PaO₂ below 60 mmHg, and in specific examples to below 40 mmHg (SaO₂=70%), which is adjacent to the steep part of the oxyhemoglobin dissociation curve and provides a large signal change and thus a more precise calculation of blood flow.

Third, Johnson uses phase contrast to measure the blood flow response to hypoxia, which is only suitable for large extracranial arteries, particularly the carotid artery and vertebral artery. In contrast, the present specification uses susceptibility imaging to measure the blood flow in the parenchyma of the brain resulting in a map of the distribution of the increase in blood flow.

Finally, in Johnson the reoxygenation was to hyperoxia (PO₂ of 110 mmHg). Such hyperoxic reoxygenation markedly prolongs the time to attaining a repeat measure of cerebral hemodynamic parameters at baseline and makes the approach less practical for clinical work where repeat measures are desirable. The method described in this application re-saturates the hemoglobin to near saturation while keeping the PO₂ at a level that readily yields to repeat rapid desaturations.

The present specification encompasses any one of the following aspects:
1) A method of measuring cerebral vascular reactivity in a subject comprising the steps of:
   (a) using sequential gas delivery to impose a first stepwise reoxygenation from a first hypoxic condition;
   (b) using sequential gas delivery to impose a second stepwise reoxygenation from a second hypoxic condition, the second hypoxic condition selected to induce greater vasodilation than the first hypoxic condition;
   (c) measuring a ΔR₂* time course in a target voxel responsive to the first and second stepwise reoxygenations;
   (d) calculating a first and second perfusion metric based on the ΔR₂* time course measured during the first and second reoxygenations, respectively; and
   (e) comparing the first perfusion metric to the second perfusion metric to determine a cerebral vascular reactivity.
2) The method of aspect 1 wherein imposing the first and second reoxygenations includes restoring normoxia in the subject.
3) The method of aspect 1 or 2 wherein imposing the first and second reoxygenations includes restoring the subject's partial arterial pressure of oxygen (PaO₂) to between 90 and 100 mmHg.
4) The method of any one of aspects 1-3 wherein the second hypoxic condition has a longer duration or lower partial arterial pressure of oxygen (PaO₂) than the first hypoxic condition.
5) The method of aspect 3 wherein the PaO₂ during the first and second hypoxic conditions is less than 60 mmHg.
6) The method of aspect 5 wherein the PaO2 during the first and second hypoxic conditions is less than 40 mmHg.
7) The method of aspect 2 further comprising: maintaining the partial arterial pressure of carbon dioxide (PaCO₂) during the performance of steps (a) and (b).
8) The method according to any one of the preceding aspects wherein calculating the first and second perfusion metrics includes fitting a sigmoid function to the ΔR₂* time course, wherein computing the perfusion metric for the target voxel is further based on the sigmoid function.
9) The method of aspect 8 wherein the first and second perfusion metrics include relative cerebral blood volume (rCBV), and computing the first and second perfusion metric comprises computing the magnitude of the sigmoid function.
10) The method of aspect 8 or 9 wherein the first and second perfusion metric include relative cerebral blood flow (rCBF), and computing the first and second perfusion metric comprises computing the maximum rate of decrease in the sigmoid function.
11) The method of any one of aspects 8-10 wherein the first and second perfusion metric include mean transit time (MTT), and the first and second perfusion metrics are calculated as MTT=rCBV/rCBF.
12) A method of measuring cerebral vascular reactivity in a subject comprising the steps of:
   (a) using sequential gas delivery to impose a first stepwise reoxygenation from a first hypoxic condition selected to minimize vasodilation;
   (b) administering a vasoactive stimulus to the subject;
   (c) using sequential gas delivery to impose a second stepwise reoxygenation from a second hypoxic condition, the second hypoxic condition selected to minimize vasodilation;
   (d) measuring a ΔR₂* time course in a target voxel responsive to the first and second stepwise reoxygenations;
   (e) calculating a first and second perfusion metric based on the ΔR₂* time course measured during the first and second reoxygenations, respectively; and
   (f) comparing the first perfusion metric to the second perfusion metric to determine a cerebral vascular reactivity.
13) The method of aspect 12 wherein the vasoactive stimulus is carbon dioxide and step (a) further includes imposing normocapnia in the subject, and step (b) further includes imposing hypercapnia in the subject.
14) The method of aspect 13 wherein imposing the first and second reoxygenations includes restoring normoxia in the subject.
15) The method according to any one of aspects 12-14 wherein calculating the first and second perfusion metrics includes fitting a sigmoid function to the ΔR₂* time course, wherein computing the perfusion metric for the target voxel is further based on the sigmoid function.
16) The method of aspect 15 wherein the perfusion metric includes relative cerebral blood volume (rCBV), and computing the perfusion metric comprises computing the magnitude of the sigmoid function.
17) The method of aspect 15 or 16 wherein the perfusion metric includes relative cerebral blood flow (rCBF), and computing the perfusion metric comprises computing the maximum rate of decrease in the sigmoid function.
18) The method of any one of aspects 15-17 wherein the perfusion metric includes mean transit time (MTT), and the perfusion metric is calculated as MTT=rCBV/rCBF.
19) A system for measuring cerebral vascular reactivity in a subject comprising:
   a sequential gas delivery device configured to:
      impose a first stepwise reoxygenation from a first hypoxic condition; and
      impose a second stepwise reoxygenation from a second hypoxic condition, the second hypoxic condition selected to induce greater vasodilation than the first hypoxic condition;
   a magnetic resonance imaging system configured to:
      measure a ΔR₂* time course in a target voxel responsive to the first and second stepwise reoxygenations;
   and
   a processor configured to:
      calculate a first and second perfusion metric based on the ΔR₂* time course measured during the first and second reoxygenations, respectively; and
      compare the first perfusion metric to the second perfusion metric to determine a cerebral vascular reactivity.
20) The system of aspect 19 wherein imposing the first and second reoxygenations includes restoring normoxia in the subject.
21) The system of aspect 19 or 20 wherein imposing the first and second reoxygenations includes restoring the subject's partial arterial pressure of oxygen (PaO₂) to between 90 and 100 mmHg.
22) The system of any one of aspects 19-21 wherein the second hypoxic condition has a longer duration or lower partial arterial pressure of oxygen (PaO₂) than the first hypoxic condition.
23) The system of aspect 21 wherein the PaO₂ during the first and second hypoxic conditions is less than 60 mmHg.
24) The system of aspect 23 wherein the PaO₂ during the first and second hypoxic conditions is less than 40 mmHg.
25) The system of aspect 20 wherein the sequential gas delivery device is further configured to maintain the partial arterial pressure of carbon dioxide (PaCO₂) while imposing the first and second stepwise oxygenations.
26) The system according to aspects 19-25 wherein calculating the first and second perfusion metrics includes fitting a sigmoid function to the ΔR₂* time course, wherein computing the perfusion metric for the target voxel is further based on the sigmoid function.
27) The system of aspect 26 wherein the first and second perfusion metrics include relative cerebral blood volume (rCBV), and computing the first and second perfusion metric comprises computing the magnitude of the sigmoid function.
28) The system of aspect 26 or 27 wherein the first and second perfusion metric include relative cerebral blood flow (rCBF), and computing the first and second perfusion metric comprises computing the maximum rate of decrease in the sigmoid function.
29) The system of any one of aspects 26-28 wherein the first and second perfusion metric include mean transit time (MTT), and the first and second perfusion metrics are calculated as MTT=rCBV/rCBF.
30) A system for measuring cerebral vascular reactivity in a subject comprising:
   a sequential gas delivery device configured to:
      impose a first stepwise reoxygenation from a first hypoxic condition selected to minimize vasodilation;
      administer a vasoactive stimulus to the subject; and
      impose a second stepwise reoxygenation from a second hypoxic condition selected to minimize vasodilation;
   a magnetic resonance imaging system configured to:
      measure a ΔR₂* time course in a target voxel responsive to the first and second stepwise reoxygenations;
   and
   a processor configured to:
      calculate a first and second perfusion metric based on the ΔR₂* time course measured during the first and second reoxygenations, respectively; and
      compare the first perfusion metric to the second perfusion metric to determine a cerebral vascular reactivity.
31) The system of aspect 30 wherein the vasoactive stimulus is carbon dioxide and administering the vasoactive stimulus includes imposing hypercapnia in the subject, and wherein imposing the second stepwise reoxygenation includes restoring normocapnia in the subject.
32) The system of aspect 31 wherein imposing the first and second reoxygenations includes restoring normoxia in the subject.
33) The system according to any one of aspects 30-32 wherein calculating the first and second perfusion metrics includes fitting a sigmoid function to the ΔR₂* time course, wherein computing the perfusion metric for the target voxel is further based on the sigmoid function.
34) The system of aspect 33 wherein the perfusion metric includes relative cerebral blood volume (rCBV), and computing the perfusion metric comprises computing the magnitude of the sigmoid function.
35) The method of aspect 33 or 34 wherein the perfusion metric includes relative cerebral blood flow (rCBF), and computing the perfusion metric comprises computing the maximum rate of decrease in the sigmoid function.
36) The method of any one of aspects 33-35 wherein the perfusion metric includes mean transit time (MTT), and the perfusion metric is calculated as MTT=rCBV/rCBF.

The specification is explained herein by way of example:

### Example 1

We recognized that the development of hypoxia on breathing hypoxic gas is prolonged due to the time it takes to dilute and wash out the oxygen remaining in the lung, called the functional residual capacity (FRC). As such, the vasodilatation in the brain cannot occur faster than the dilution of the FRC by breathing hypoxic gas. We hypothesized that faster washout of FRC will cause faster hypoxia and faster cerebral vasodilation. As such we used the principles of sequential gas delivery (described herein with respect to FIG. 3) to administer 4% oxygen in 96% nitrogen in a controlled manner using a prospective targeting gas blender (RespirAct™, Toronto Canada) to minimize the time to attain an arterial PO₂ of 40 mmHg within about 4-5 breaths. We studied the middle and posterior cerebral artery blood flow velocity (a surrogate of blood flow) as measured by Transcranial Doppler, during rapid reductions in lung oxygenation in 24 healthy volunteers. We succeeded in reducing the lung PO₂ in less time than the vasodilatory response, leaving the vasodilatory response as the temporal limiting factor. We then studied the time course of vasodilation response to hypoxia as described below. These unique findings are the basis of this application.

### Study of Temporal Course of Response of Cerebral Arteries to Hypoxia

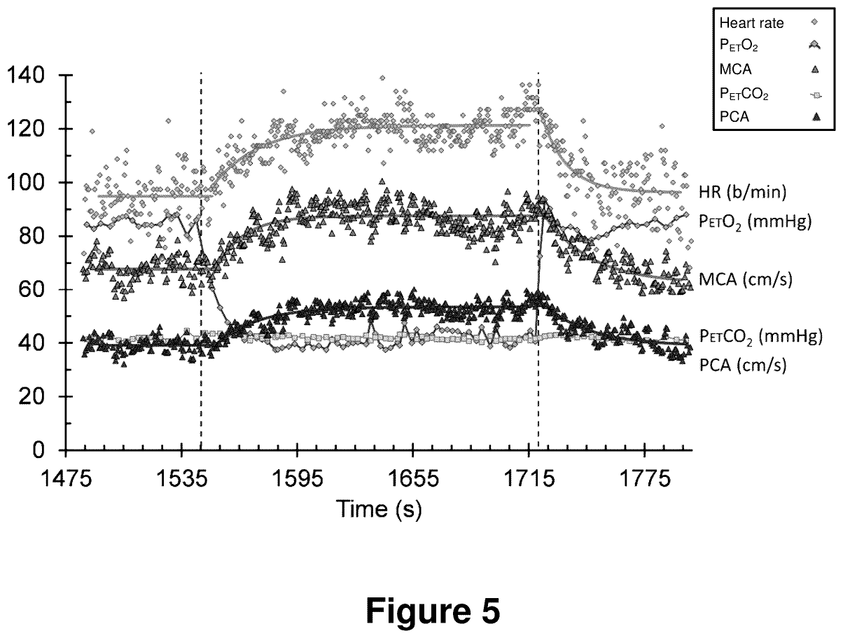

After obtaining written informed consent of 14 (6 F) healthy non-smoking subjects of mean (SD) age 28.2 (8) years were recruited for the study. Subjects were fitted with a face mask and connected to a sequential gas delivery device which targeted end-expired PO₂ and PCO₂ (RespirAct™ Thornhill Medical, Toronto Canada). The device has been shown to operate such that the end-tidal values of PO₂ and PCO₂ are equal to the arterial corresponding arterial values. Middle and posterior cerebral artery flow velocities were measured using trans-cranial Doppler (Delica EMS-9D Pro, Shenzhen, 518107, P.R. China) at 2 MHz and sampled at 125 Hz. A typical example of the response is shown in FIG. 5. We found that vasodilation was a decreasing exponential function with the maximal vasodilation occurring in about 60 s with a time constant of about 20 s.

FIG. 5 is a graph showing an example of trans cranial doppler (TCD) velocity responses to hypoxia. The dashed lines mark the onset and offset of hypoxia, with breath-by-breath values of partial pressures of end tidal O₂ and CO₂. Note the abrupt drop in PO₂ over about 10 s, with most of the decline in 5 s. The time constant of the middle cerebral artery (MCA) response is about 20 s. Other values in figure legend.

In people who tend to have small FRC such as short, thin adults, children, females, it is possible to reach PO₂ 40 within about 3 large breaths in about 6 s. The brief time period to develop hypoxia in the lungs may result in minimal vasodilation in cerebral arteries. The hemodynamic measures made during the reoxygenation phase will therefore reflect baseline cerebral blood flow. If hypoxia at PO₂ of about 40 mmHg is sustained for about 60 s or longer, the hemodynamic measures from the sudden reoxygenation phase will reflect stimulated flow. Thus, the differences in flow will reflect CVR.

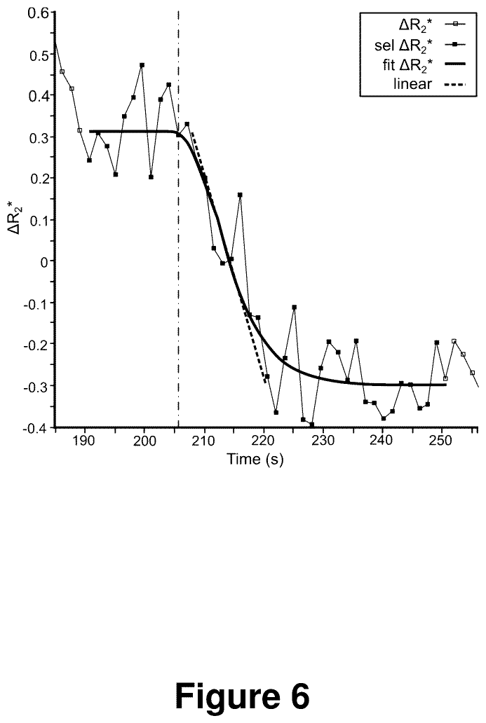

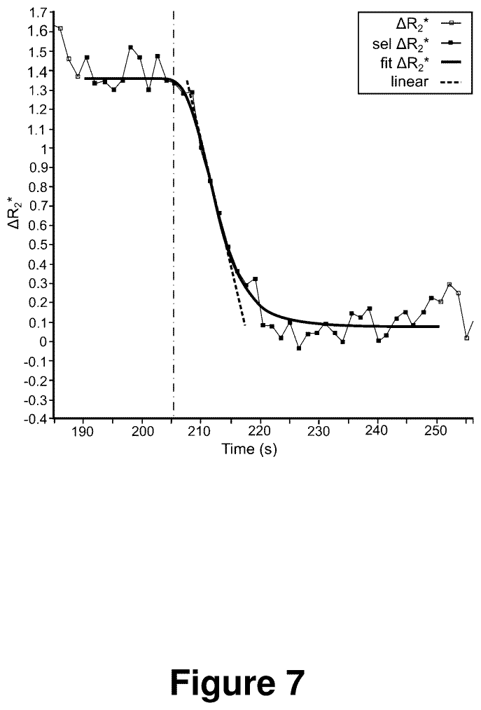

FIG. 6 and FIG. 7 are graphs showing the ΔR₂* signal during revascularization in 2 voxels with different levels of noise. FIG. 6 is representative of a noisy signal, with FIG. 7 is representative of a less noisy signal. The solid line is the fit of the Gompertz function to the ΔR₂* signal response to a stepwise reoxygenation. The dashed line is superimposed on the linear portion of the function and extended from baseline (top) and asymptote (bottom). The vertical, dot-dash line is the reference time cursor used for all voxels to calculate blood arrival time (BAT).

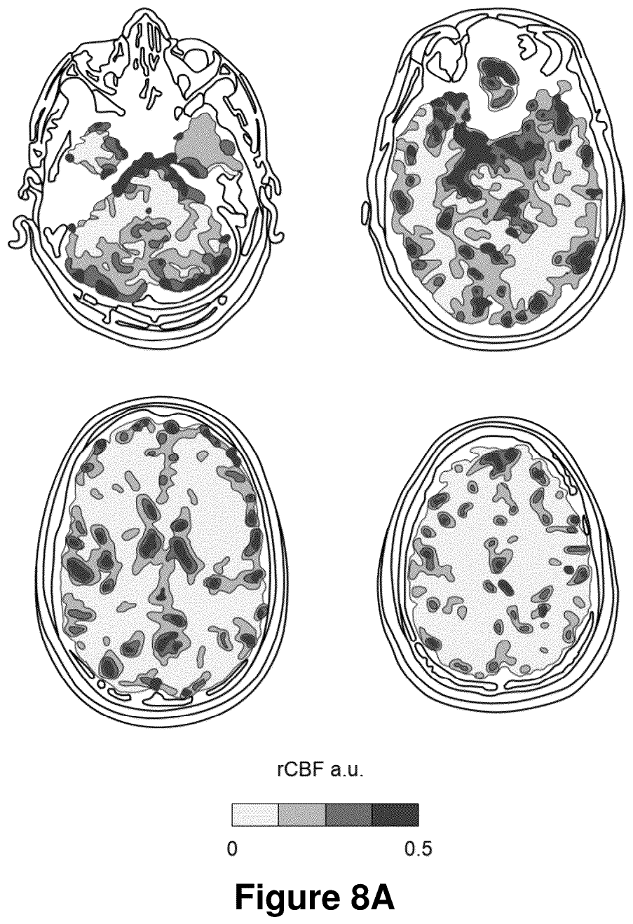

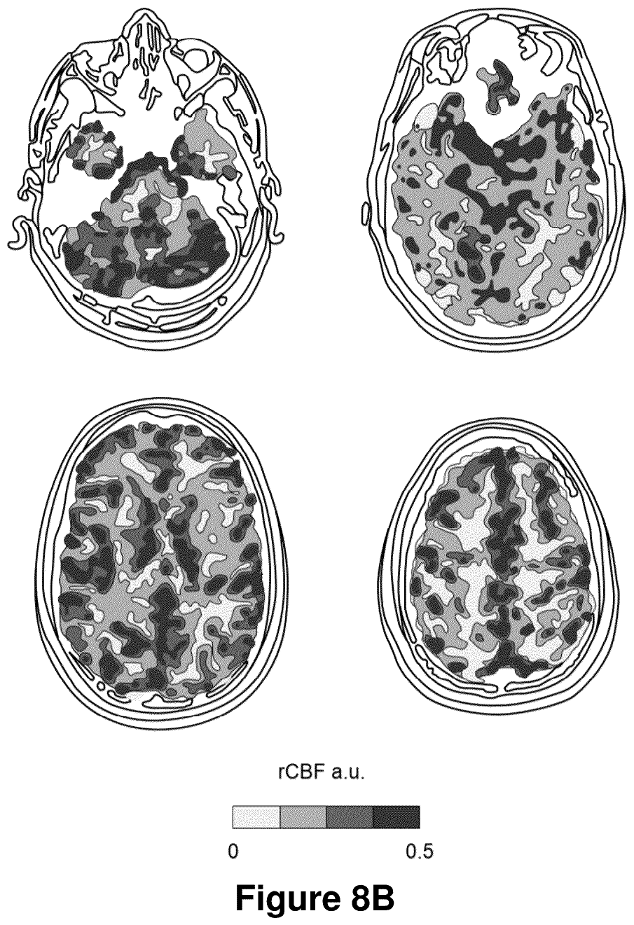

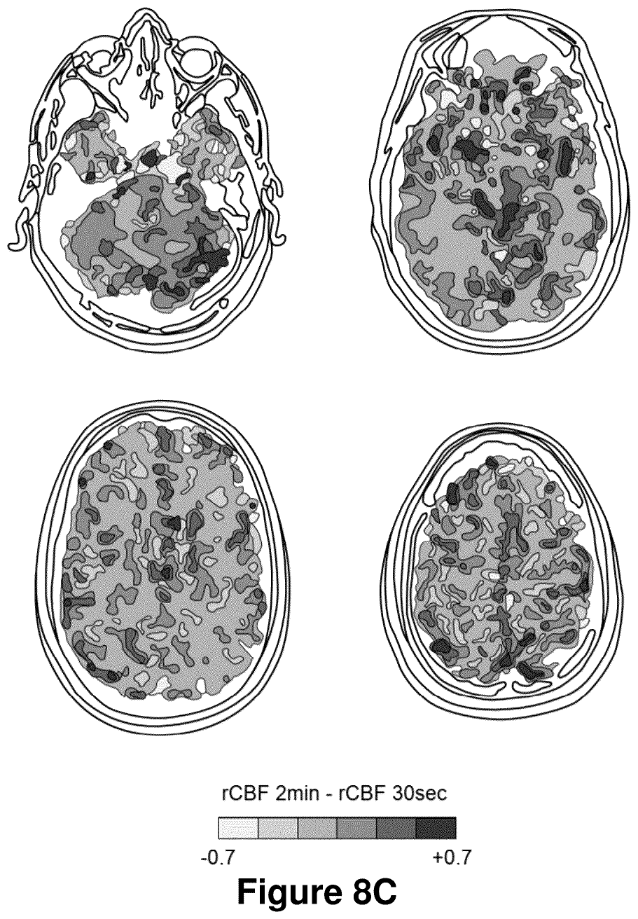

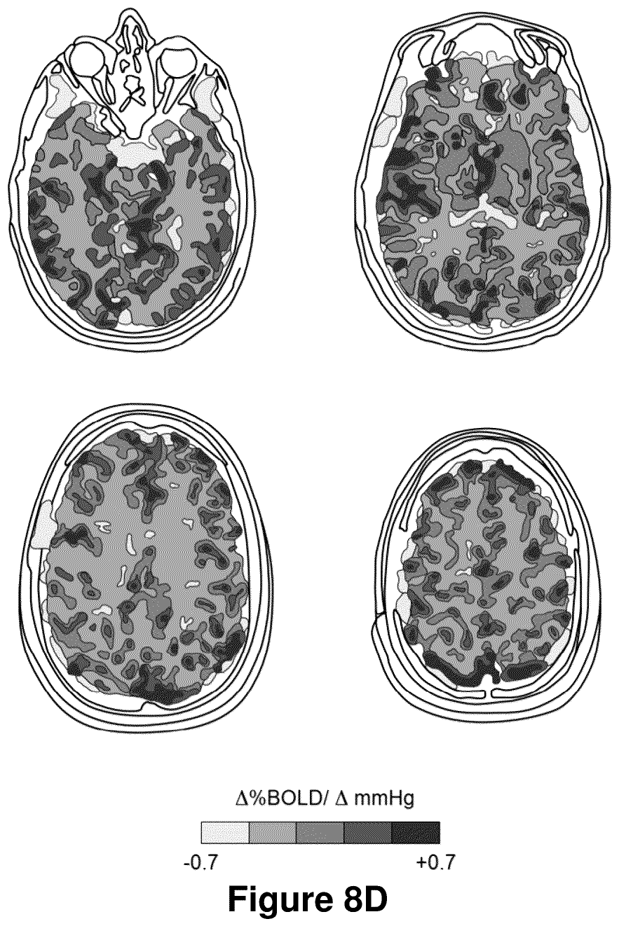

FIGS. 8A to 8D are perfusion maps obtained through exemplary performance of method 200. A) shows the CBF_{baseline} obtained from imposing a first hypoxic condition of 30 s. B) shows the CBFₛₜᵢₘ obtained from imposing a second hypoxic condition of 2 minutes. C) shows the CVR calculated as voxelwise subtraction of CBFₛₜᵢₘ−CBF_{baseline}. By comparison, D) shows the CVR calculated as ΔBOLD/ΔPCO₂ using hypercapnic stimulus using color scale for relative changes.

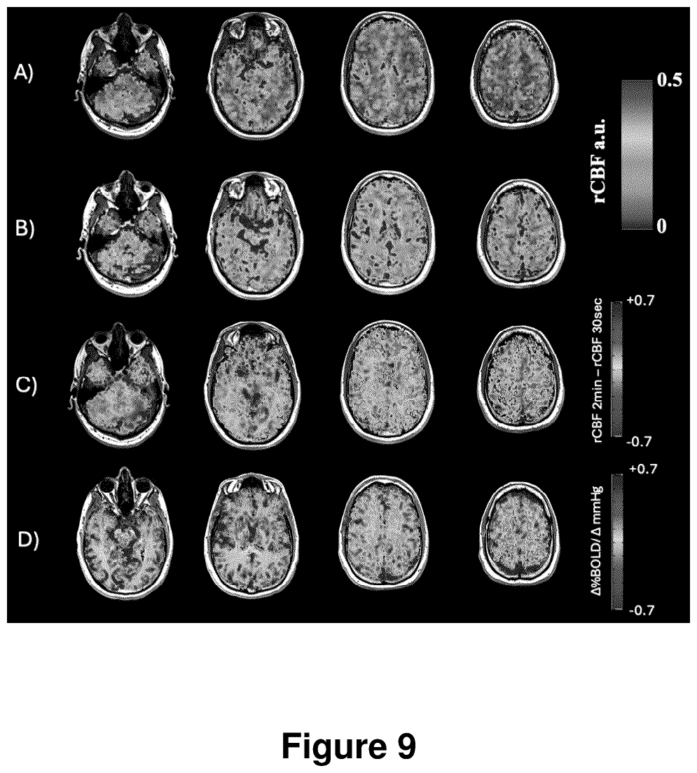

FIGS. 9A to 9D are perfusion maps showing the results of FIGS. 8A to 8D in a color gradient.

The many features and advantages of the invention are apparent from the detailed specification and, thus, it is intended by the appended claims to cover all such features and advantages of the invention that fall within the true spirit and scope of the invention. Further, since numerous modifications and changes will readily occur to those skilled in the art, it is not desired to limit the invention to the exact construction and operation illustrated and described, and accordingly all suitable modifications and equivalents may be resorted to, falling within the scope of the invention.

What is claimed is:

1. A method of measuring cerebral vascular reactivity in a subject comprising the steps of:
(a) using sequential gas delivery to impose a first stepwise reoxygenation from a first hypoxic condition;
(b) using sequential gas delivery to impose a second stepwise reoxygenation from a second hypoxic condition, the second hypoxic condition selected to induce greater vasodilation than the first hypoxic condition;
(c) measuring a ΔR₂* time course in a target voxel responsive to the first and second stepwise reoxygenations;
(d) calculating a first and second perfusion metric based on the ΔR₂* time course measured during the first and second reoxygenations, respectively; and
(e) comparing the first perfusion metric to the second perfusion metric to determine a cerebral vascular reactivity.

2. The method of claim 1 wherein imposing the first and second reoxygenations includes restoring normoxia in the subject.

3. The method of claim 1 wherein imposing the first and second reoxygenations includes restoring the subject's partial arterial pressure of oxygen (PaO₂) to between 90 and 100 mmHg.

4. The method of claim 1 wherein the second hypoxic condition has a longer duration or lower partial arterial pressure of oxygen (PaO₂) than the first hypoxic condition.

5. The method of claim 3 wherein the PaO₂ during the first and second hypoxic conditions is less than 60 mmHg.

6. The method of claim 5 wherein the PaO2 during the first and second hypoxic conditions is less than 40 mmHg.

7. The method of claim 2 further comprising: maintaining the partial arterial pressure of carbon dioxide (PaCO₂) during the performance of steps (a) and (b).

8. The method according to claim 1 wherein calculating the first and second perfusion metrics includes fitting a sigmoid function to the ΔR₂* time course, wherein computing the perfusion metric for the target voxel is further based on the sigmoid function.

9. The method of claim 8 wherein the first and second perfusion metrics include relative cerebral blood volume (rCBV), and computing the first and second perfusion metric comprises computing the magnitude of the sigmoid function.

10. The method of claim 8 wherein the first and second perfusion metric include relative cerebral blood flow (rCBF), and computing the first and second perfusion metric comprises computing the maximum rate of decrease in the sigmoid function.

11. The method of claim 8 wherein the first and second perfusion metric include mean transit time (MTT), and the first and second perfusion metrics are calculated as MTT=rCBV/rCBF.

12. A method of measuring cerebral vascular reactivity in a subject comprising the steps of:
(a) using sequential gas delivery to impose a first stepwise reoxygenation from a first hypoxic condition selected to minimize vasodilation;
(b) administering a vasoactive stimulus to the subject;
(c) using sequential gas delivery to impose a second stepwise reoxygenation from a second hypoxic condition, the second hypoxic condition selected to minimize vasodilation;
(d) measuring a ΔR₂* time course in a target voxel responsive to the first and second stepwise reoxygenations;
(e) calculating a first and second perfusion metric based on the ΔR₂* time course measured during the first and second reoxygenations, respectively; and
(d) comparing the first perfusion metric to the second perfusion metric to determine a cerebral vascular reactivity.

13. The method of claim 12 wherein the vasoactive stimulus is carbon dioxide and step (a) further includes imposing normocapnia in the subject, and step (b) further includes imposing hypercapnia in the subject.

14. The method of claim 13 wherein imposing the first and second reoxygenations includes restoring normoxia in the subject.

15. The method of claim 12 wherein calculating the first and second perfusion metrics includes fitting a sigmoid function to the ΔR₂* time course, wherein computing the perfusion metric for the target voxel is further based on the sigmoid function.

16. The method of claim 15 wherein the perfusion metric includes relative cerebral blood volume (rCBV), and computing the perfusion metric comprises computing the magnitude of the sigmoid function.

17. The method of claim 15 wherein the perfusion metric includes relative cerebral blood flow (rCBF), and computing the perfusion metric comprises computing the maximum rate of decrease in the sigmoid function.

18. The method of claim 15 wherein the perfusion metric includes mean transit time (MTT), and the perfusion metric is calculated as MTT=rCBV/rCBF.
